(19) United States
(12) Patent Application Publication
(10) Pub. No.: US 2003/0008830 A1
(43) Pub. Date: Jan. 9, 2003

(54) Composition and method for treating burns and regenerating burn wound tissue

(76) Inventor: Luzdalma C. Prozillo, Cambui (BR)

Correspondence Address: MICHAEL C. CESARANO, SUNTRUST INTERNATIONAL CENTER, 28TH FLOOR, 1 S.E. 3RD AVENUE, MIAMI, FL, 33131-1714, US

(21) Appl. No.: 09/887,940

(22) Filed: Jun. 22, 2001

(51) Int. Cl. A61K 38/39; C07K 14/78

(52) U.S. Cl. 514/21; 530/356

(57) ABSTRACT

Gel wound dressings especially useful for treating burns. The Gel is obtained by the partial hydrolysis of collagen plus addition of sulfated galactose monomers, pectin and hydroxyethylcellulose The gel is characterized by insolubility in alcohol, cool water and most other organic solvents, with high capacity of hydro absorption. It is hygroscopic, not antigenic, of uniform porosity with physical aspect transparency.

### KNOWN PRIOR ART

U.S. Pat. No. 3,157,524 November 1964 Artandi

U.S. Pat. No. 3,742,955 July 1973 Battista et al.

U.S. Pat. No. 3,939,831 February 1976 Cioca et al.

U.S. Pat. No. 4,070,459 November 1978 Huber et al.

U.S. Pat. No. 4,210,721 July 1980 Monschimer et al.

U.S. Pat. No. 4,243,582 June 1981 Spilburg et al.

U.S. Pat. No. 4,789,663 June 1988 Wallace et al.

U.S. Pat. No. 4,959,238 September 1990 Hall, et al.

U.S. Pat. No. 5,073,378 December 1991 Shoshan et al.

U.S. Pat. No. 6,046,160 April 2000 Obi-Tabot

### FIELD OF THE INVENTION

The invention relates to the field of regeneration of burn wounds on skin. More specifically, this invention relates to medicinal dressings that provide an advanced healing environment for burn wounds. Good wound healing is characterized by rapid and complete regeneration of the damaged tissue. Considerable efforts have been expended in the study of wound dressings with the aims of finding which dressings are most effective in promoting wound healing. The process of wound healing is complex and is not fully understood. High macrophage activity is desirable, particularly during the early stages of healing, to kill bacteria and to remove cell debris and foreign matter. This activity is generally accompanied by inflammation. High fibroblast activity is desirable, particularly during the later stages of healing, to produce a high rate of regeneration. The invention relates to a method to regenerate the skin defects caused by burn injuries of first and second degree burns. The treatment involves application of a hydrolyzed collagen gel to the affected area. The gel is a reconstituted material derived from collagen hydrolysis.

### BACKGROUND OF THE INVENTION

Collagen dressing has been made heretofore by a number of processes and have been found to be advantageous a skin dressing or skin plasters particularly suitable for the treatment of burn. See for example, U.S. Pat. Nos. 4,210,721, 6,046,160 and British Pat. No. 942,226.

The process of wound healing consists of three phases during which the injured tissue is repaired, regenerated, and new tissue is reorganized into a scar. These three phases are classified as: a) an inflammation phase which begins from day 0 to 3 days, b) a cellular proliferation phase from 3 to 12 days, and c) a remodeling phase from 3 days to about 6 months. In all three phases, antioxidants play a vital role in the healing process.

In the inflammation phase, inflammatory cells, mostly neutrophils, enter the site of the wound followed by lymphocytes, monocytes, and later macrophages. The neutrophils that are stimulated begin to release proteases and reactive oxygen species into the surrounding medium with potential adverse effects on both the adjacent tissues and the invading microorganisms.

The proliferative phase consists of laying down new granulation tissue, and the formation of new blood vessels in the injured area. The fibroblasts, endothelial cells, and epithelial cells migrate in the wound site. These fibroblasts produce the collagen that is necessary for wound repair. Ascorbic acid is crucial in the formation of collagen. A decrease of ascorbic acid at the injury area will decrease the rate of wound healing.

In reepithelialization, epithelial cells migrate from the free edges of the tissue across the wound. This event is succeeded by the proliferation of epithelial cells at the periphery of the wound. Research has also shown that reepithelialization is enhanced by the presence of occlusive wound dressings which maintain a moisture barrier.

The final phase of wound healing, which is remodeling, is effected by both the replacement of granulation tissue with collagen and elastin fibers and the devascularization of the granulation tissue. Recent studies have shown that topical application of antioxidants, especially alpha-tocopherol, reduces scarring and normalizes blood coagulation during therapy.

As described in U.S. Pat. No. 3,812,252, a particularly effective healing treatment for wounds and skin defects such as burns, ulcers and lesions is the application of a medicinal dressing containing as an essential ingredient starch hydrolysate having dextrose e5quivalent of less than about 35. In such wound treatment the starch hydrolysate produces the formation of a film which is intimately adhered to the underlying granulation tissue and which is semi-permeable to gas and fluids and provides an ideal protective cover that will reduce fluid and plasma losses and invasion by pathogenic bacteria. In addition, it appears that the starch hydrolysate provides a topical or local hyperalimentation, that is local nutrition, providing a gradual release of glucose which is particularly effective in nutrition of tissue, both damaged and nascent, which have become relatively isolated from normal blood flow nutrition. The cessation of blood flow to such an ischemic lesion can be developed in a slow and gradual form such as in the case of decubitus ulcers and stasis ulcers, or may take place more acutely such as in thermo-radiation and chemical burns. In the absence of nutrition, the rate of fluid delivery of nutrients decreases bringing a progressive impairment in the viability of cells and tissues. This eventually leads to degeneration and death of the tissue and cells in a condition known as necrosis. Necrosis is generally accompanied by bacterial, fungal and/or viral contamination. As further pointed out in the aforementioned patent, treatment of exudative skin wounds with a starch hydrolysate dressing produces a greatly reduced bacteria count of an infected wound and inhibits infection of an uninfected wound. In addition, application of the starch hydrolysate to a wound or ulcer produces a film or semi-permeable membrane which allows edematous liquid to pass through while proteinaceous material is retained within the body, allowing reduction in the volume of exudate in relatively clean condition. The healing process is brought about by complex biological mechanisms generally involving several groups of special cells and proteins. Leukocytes, such as neutrophils and macrophages, crown the wound site and digest foreign pathogens and debris. Such cells also send out chemical signals that marshal fibroblasts in the wound vicinity and ultimately generate connective structures, principally, collagen, which make up a major portion of the new tissues. Endothelial cells generate new blood capillaries that grow into the reconstructed tissue areas where their presence is necessary to supply nutrients to the newly growing tissue cells and remove catabolic products. As the new capillaries grow, the cells on the margin of the wound simultaneously multiply and grow inwardly. The fibrous tissue arising from this cell growth eventually fills the wound cavity with a network of interlacing threads of collagen which in due time, arrange themselves in firm bands and form the permanent new tissue.

The surface of the wound is subsequently covered by processes of enlargement, flattening, and multiplication of the epithelial cells at the wounds' edge. These epithelial cells spread as sheets into the wound, beneath the scab. Eventually the proliferating epithelial cell sheets emanating from the wound sides coalesce to cover and close the wound on the outer surface. Until such time as at least superficial healing has occurred, or healing is impaired, the individual remains at risk from continued or new infection. Hence, there is a time versus rate related risk factor involved in all wound situations. The quicker the wound can heal, the sooner the risk is removed.

Known collagen dressings are obtained usually by an extraction process from fibrous insoluble protein collagen, which is widely found in nature being the major constituent of skin, bones and connective tissue. Process include steps in which the protein materials are treated in water with calcium hydroxide and sharpening agents. The caustic materials remove minerals and soluble proteins from the bones and caustic material remove minerals and soluble proteins from the bones and cause the hide to swell.

In accordance with this invention, improvements in the treatment of wounds have been developed to provide dressing for wound treatment, effective to promote the healing process.

### SUMMARY OF THE INVENTION

A composition for use in promoting the healing of surface burn wounds, comprising a pure bovine skin collagen in admixture with sulfated galactose monomers, pectin and hydroxyethylcellulose, wherein the bovine collagen is purified from animal hide and is relatively insoluble in alcohol, cool water and other organic solvents.

The gel is obtained submitting this material to an alkaline pre pretreatment, pH 4/8, followed by hot water extraction 3 minutes at 40° C., resulting in a gel with an isoelectric point of approximately 5.0.

The gel for being a hydrocolloid of origin protein character anphotero, associated to the presence of groups amine and carboxyl in the amino acids. In the conversion of the collagen to gel. The process of controlled hydrolysis is used to obtain the disorganization of the helical structure (bridges of hydrogen H), hydrolysis of connections interpolypeptides and intrapolypeptides (thermal denaturation of the collagen) and disorganization of the helical structure.

Some of the amino acids in the structure, peptides, contain functional groups that are hydrolyzed under special conditions, taking to changes in the swinging of the groups loaded of the molecules being considered the form and size of the molecule of the material.

Biochemically, the gel is formed from procollagen transformed to collagen monomer, which is laid down in tissue as mature collagen. The mature insoluble collagen is hydrolyzed to form gel. The product resulting is heterogeneous protein mixture of polypeptide chains, composed of 18 amino acids in connection with polypeptides in the formation of the molecule, with a unique sequence of amino acids, which is decisive for functional properties, the structure is very well ordinate and linked in fibrilles that form the constituent fibers of the fabric connective in the group.

This basic unit of amino acids is formed of three molecules polypeptide arranged in helix form. All collagen contain regions at each end, which do not have the triple glycine sequence and are this not helical theses regions are thought to be responsible for the immunogenic, condition associated with most collagen preparations.

### BRIEF DESCRIPTION OF THE DRAWINGS

FIG. 1 illustrates the relative proportions of the various components of the gel of this invention.

FIG. 2 depicts a structural chain showing the molecular structure of the gel of this invention.

### DETAILED DESCRIPTION OF THE INVENTION

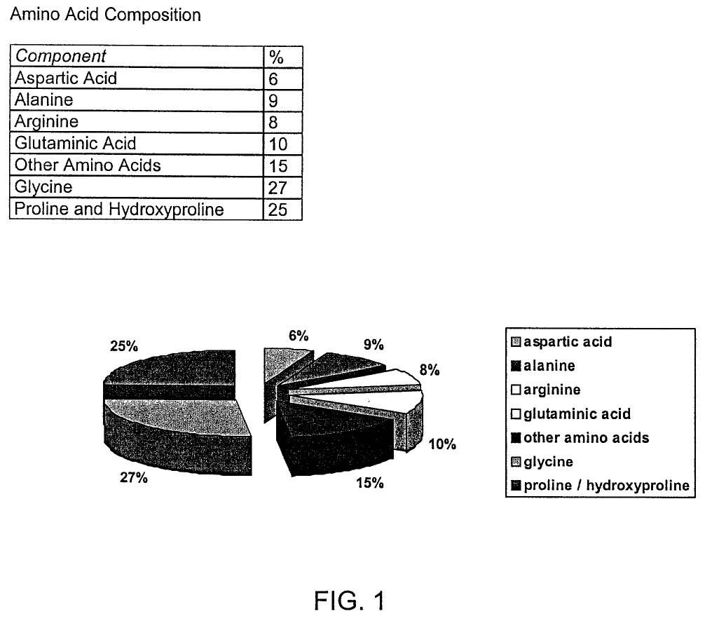

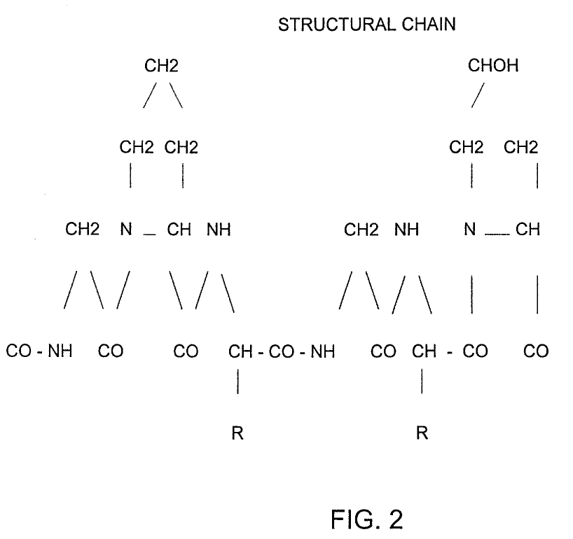

The gel of this invention is prepared by the series of steps set out below. The gel preferably has the physical and chemical properties shown and depicted in FIGS. 1 and 2, and described below.

The gel has its peculiar characteristic in the high glycine content, hydroxyproline and hydroxylisine, constituted of 84-86% of protein, 9-12% of water and 1-3% of mineral salts. Gel molecules contain repeating sequences of glycine-X-Y triples, where X and Y are frequently proline and hydroxyproline amino acids. These sequences are responsible for the triple helical structure and its ability to form gels where helical regions form chains immobilizing water.

The steps used in the manufacture of the gel described herein include the following:

Hide preparation

Treatment of collagen

Extraction

Purification

Concentration

Sterilization

Cooling

Drying

Composition of the gel:

Aminogram: 880010

Protein: 79,3800%

Volume Hydrolyzed: 0,0629 g

| Component | GAA 100 g | GAA 16 N |
|---|---|---|
| Aspartic Acid | 33,6700 | 42,4100 |
| Threonine | 1,3100 | 1,6500 |
| Serine | 2,4600 | 3,0900 |
| Glutaminic Acid | 7,8200 | 9,8500 |
| Proline | 8,3900 | 10,5300 |
| Glycine | 18,2600 | 23,0000 |
| Alanine | 0,6300 | 0,7900 |
| Valine | 1,2600 | 1,5800 |
| Methionine | 0,2200 | 0,2800 |
| Isoleucine | 0,9600 | 1,2100 |
| Leucine | 1,7800 | 2,2400 |
| Tyrosyne | 0,2200 | 2800 |
| Phenilanine | 1,0700 | 13400 |
| Histidine | 0,5100 | 6400 |
| Lysine | 2,0900 | 26300 |
| Arginine | 4,0500 | 5,1000 |

Variations of the Formula

1)

| | |
|---|---|
| Gel | 3,000% |
| Acido Hialuronico | 5,000% |
| Chlorophyceae | 2,000% |
| Decarboxylcarnosine | 0,500% |
| Sulfated galactose monomers | 50,000 g |

2)

| | |
|---|---|
| Gel | 3,000% |
| Acid Hialuronico | 5,000% |
| Chlorophyceae | 2,000% |
| Decarboxylcarnosine | 0,500% |
| Calcium Alginate | 50,000 g |

3)

| | |
|---|---|
| Gel | 3,000% |
| Acid hialuronico | 5,000% |
| Decarboxylcarnosine | 0,500% |
| Hydroxyethylcellulose | 50,000 g |

4)

| | |
|---|---|
| Gel | 3,000% |
| Acid hialuronico | 5,000% |
| Decarboxylcarnosine | 0,500% |
| Pectin | 0,100% |
| Hydroxyethylcellulose | 50,000 g |

Steps Used in Production

1—Skin Reception and Handling:

The raw material used is bovine hide. The skin is inspected and transported to the cutters to reduce the skin size according to process specifications. After cutting the skin is transported to the pits for the alkaline treatment.

2—Alkaline Treatment:

The process consists of an initial treatment to remove fat and albuminous proteins, which also promotes the breakage of secondary bonds of collagen allowing gel formation. At the same time that the albuminous proteins are eliminated the fat is saponified. Through this process that is conducted in liming pits the raw material is treated at pH 11.0-13.5, during a period up 2 to 12 days, depending on the raw material and treatment temperature. Is used intermittently stirred compressed air to prevent the development of micro-organisms.

3—Acid Treatment:

The material is submitted to 30-50 hours in washing vessels eliminating the alkali as well non-collagenic material, resulting in a adequate pH point for the gelatins extraction.

4—Extraction:

The gel is extracted using hot water jet into stainless steel tanks.

5—Centrifugation:

The solution obtained is centrifugate to remove exiting fat and fibers, than the solution is submitted a mechanical filtration in a cellulose filter.

6—Ultra Filtration:

A porous membrane system is used to dewater the solution and remove some salts.

7—Anionic exchanger:

The solution is submitted to an anionic exchange to remove the ash of remaining salts

8—Filtration:

The solution is filtered one more time on cellulose filters.

9—Evaporation:

The solution is submitted to a low temperature evaporation process.

10—Sterilizer:

The concentrated solution is submitted to a temperature of 140/145° C. during 5 to 6 seconds.

11—Additions:

Adjusting the composition pH, some materials are added like SO2 and Peroxide CCP.

12—Cooling:

The solution is submitted to a thermal exchange to prevent the molecular rearrangement of the collagenous gel.

Industrial Applicability

The compositions and methods described herein are useful in the treatment of burn areas and the regeneration of skin on areas having first and second degree burns. The compositions aid in the regrowth and healing of the affected areas.

I claim as my invention:

1. A composition for use in promoting the healing of surface burn wounds, comprising a pure bovine skin collagen gel in admixture with sulfated galactose monomers, pectin and hydroxyethylcellulose, wherein the collagen is purified from animal hide and is relatively insoluble in alcohol, cool water and other organic solvents.

2. A method of preparing collagen gel useful in treatment of burn wounds comprising the steps of:
subjecting animal hide pieces to an alkaline treatment at pH of 11.0-13.5 for a period of two to twelve days to form a partially hydrolyzed biomass;
treating said biomass in an acid bath;
extracting gel from said biomass with hot water;
centrifuging said gel;
dewatering said gel; and
drying said gel at a relatively low temperature.

3. The method of claim 2 and sterilizing said gel at about 140-145 degrees C. for 5 to 6 seconds.

4. The method of claim 2 wherein said gel has a high glycent, hydroxyprolent and hydroxylisine content and comprises 84-86% protein, 9-12% water and 1-3% mineral salts.

5. The composition of claim 1 wherein said gel has a high glycent, hydroxyprolent and hydroxylisine content and comprises 84-86% protein, 9-12% water and 1-3% mineral salts.
